(19) United States
(12) Patent Application Publication
(10) Pub. No.: US 2010/0036466 A1
(43) Pub. Date: Feb. 11, 2010

(54) LEAD CONSTRUCTION WITH COMPOSITE MATERIAL SHIELD LAYER

(75) Inventors: Xiaoyi Min, Thousand Oaks, CA (US); Yong D. Zhao, Simi Valley, CA (US); Pedro Medrano, Norcross, GA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/189,555

(22) Filed: Aug. 11, 2008

(51) Int. Cl. A61N 1/00

(52) U.S. Cl. 607/116

(57) ABSTRACT

A lead construction includes a lead body, an electrically conductive element disposed therein, and a shield layer disposed over the conductive element formed from a composite material comprising a polymer material and a non-ferrous particulate material. The non-ferrous material can include gold, platinum, iridium, nickel, cobalt, chromium, molybdenum, carbon/graphite powders, and alloys thereof. The composite material has a non-ferrous particulate content of from about 40 to 90 volume percent, and the shield layer has a thickness of from about 0.1 to 1 mm. The composite material forms an electrically conductive layer when exposed to RF having a frequency of greater than about 64 MHz. A layer of insulating material may be interposed between the shield layer and the conductive element. The shield layer can be part of the lead body, can be an intermediate layer within the lead body, or can be an outer surface of the lead body.

### FIELD OF THE INVENTION

This invention relates to a lead construction and, more particularly, to a lead construction comprising a shield layer made from a composite material that is specially formulated to minimize and/or eliminate unwanted heating of one or more conductive elements disposed therein when exposed to radio frequency signals while still retaining desired electrical and mechanical properties, and also without adversely increasing the size of the lead construction.

### BACKGROUND OF THE INVENTION

Many implanted medical devices that are powered by electrical energy have been developed. Most of these devices comprise a power source, one or more conductors, and a load. When a patient with one of these implanted devices is subjected to high intensity magnetic fields, and radio frequency (RF) signals, such as those used during magnetic resonance imaging (MRI) procedures, such implanted devices can be subject to heating caused by the RF signals. In a particular example, where the implanted medical device is provided in the form of one or more conductive leads used in conjunction with a cardiac pacing device, it is known that conventional conductive leads are subject to unwanted RF induced heating during the MRI procedure.

Conventional leads used for cardiac pacing applications typically comprise one or more conductive elements or conductors that are used to send an electronic pacing signal from a cardiac pacing device to an electrode adjacent an end of the lead, and may also include a further conductor used to provide a signal back to the pacing device. The conductor or conductors are typically covered with an insulator material provided in the form of a sheath or tube made from a material that is relatively biocompatible to insulate the conductor from human tissue. Example insulating materials used for such cardiac leads are polymeric materials well known in the art that include silicone materials, polyurethane materials, and combinations thereof.

During the MRI procedure, the insulator material does not function to shield the conductor or conductors from the RF signals or energy, which can cause intense heating along the length of the conductor, and at the electrode or electrodes that are positioned or attached adjacent the person's heart tissue, e.g., along the heart wall. Accordingly, such RF induced heating of a conventional lead can cause unwanted injury to a person having such implanted device during the MRI procedure.

Additionally, RF energy that is inductively coupled into an electrically conductive lead during an MRI procedure can induce unwanted voltages into the lead that can adversely interfere with the desired pacing voltages, and that can cause unwanted stimulations to the heart.

Attempts in the art that have been made to address unwanted the impact of the MRI procedure on implanted medical devices include those that have focused on the electromagnetic fields induced by the MRI. Such attempts have included developing circuitry in the implanted medical device to limit and/or control MRI induced voltage surges. Such attempts both add to the size and packaging of the implanted medical device, and do not address the unwanted RF induced heating that occurs during the MRI procedure as discussed above.

Other attempts that have made to address the unwanted impact of the MRI procedure on implanted medical devices have focused on providing a magnetic shield layer over or around the electrically conductive element, e.g., around a conductor of a lead. Such attempts have focused on using a nanomagnetic particle material that is a magnetic material, i.e., iron materials and iron compounds or alloys, for forming such a shield layer that operates to deflect electromagnetic fields while remaining electrically non-conductive. While such attempts may possibly operate to reduce or control the electromagnetic impact that the MRI procedure may have on an implanted medical device, it is not clear whether such attempts also operate to effectively reduce or control the RF impact of unwanted heating.

It is, therefore, desired that an implanted medical device be constructed in a manner that operates to reduce and/or eliminate the unwanted effect of RF induced heating that can occur when subjected to an MRI procedure. It is further desired that the implanted medical device be constructed in a manner that does not adversely impact the size and/or packaging of the device. It is further desired that the device be constructed in a manner that does not adversely impact other desired electrical and/or mechanical performance properties of the device, such as the flexibility, structural integrity, long-term fatigue, and the like, e.g., when the device is provided in the form of a lead for cardiac pacing or the like.

### SUMMARY OF THE INVENTION

In one aspect, the lead construction can comprise a lead body and one or more conductive elements disposed within the lead construction. The construction further includes a shield layer disposed over at least one of the one or more conductive elements. The shield layer is formed from a composite material comprising a polymer material and a non-ferrous particulate material. The non-ferrous material can include gold, platinum, iridium, nickel, cobalt, chromium, molybdenum, carbon/graphite powders, and alloys thereof.

In an example embodiment, the composite material comprises a volume content of the non-ferrous particulate material sufficient to form an electrically conductive layer at RF frequencies greater than about 64 MHz, and does not form an electrically conductive layer at RF frequencies that are lower than about 64 MHz. In an example embodiment, the composite material has a non-ferrous particulate content in the range of from about 40 to 90 volume percent, and the shield layer has a thickness in the range of from about 0.1 to 1 mm.

The construction can include a layer of insulating material interposed between the shield layer and the at least one or more conductive elements. The shield layer can be provided as part of the lead body disposed around the one or more conductive elements, and may form an intermediate layer within the lead body or may form an outer surface of the lead body.

The lead construction can be provided in the form of a cardiac lead comprising one or more electrodes positioned adjacent a distal end and connected with the one or more conductive elements disposed within the lead body.

In another aspect, the lead construction comprises a tubular construction defined by distal and proximal body ends, and the lead body is formed from a polymeric material. The lead construction may comprise one or more conductive elements disposed within the lead body. A shield layer is positioned over at least one of the one or more conductive elements. The shield layer is formed from a composite material that is substantially free of a ferrous metal and that forms an electrically conductive layer when exposed to RF having a frequency of greater than about 64 MHz. The lead construction further includes an electrically insulating layer interposed between the at least one or more conductive elements and the shield layer. The shield layer can be part of the lead body, can occupy a region of the lead body that is positioned along an outer surface of the lead body, or is positioned intermediate the electrically insulating layer and a further layer of insulating material.

In another aspect a method of making a lead construction comprising the step of forming a lead body from a polymeric material to cover one or more electrically conductive elements disposed therein. Forming an electrically insulating layer over the one or more electrically conductive elements, and forming a shield layer over at least one of the one or more electrically conductive elements. The shield layer being formed from a composite material comprising a polymeric material and a non-ferrous material, and the composite material is substantially nonmagnetic. The shield layer can be position to form an outer surface of the lead body, or an inner region of the lead body.

The shield layer can be part of the lead body and the steps of forming the lead body and forming the shield layer are done separately or simultaneously. Further, the electrically insulating layer can be part of the lead body, and the steps of forming the lead body and forming the electrically insulating layer are done simultaneously.

Such lead constructions reduce or eliminate unwanted RF induced heating of the electrically conductive elements disposed within the lead without sacrificing other desired electrical and/or mechanical performance properties such as electrical insulation, mechanical stiffness, flexibility, structural integrity, and long term fatigue.

### BRIEF DESCRIPTION OF THE DRAWINGS'

These and other features and advantages of the present invention will be appreciated as the same becomes better understood by reference to the following detailed description when considered in connection with the accompanying drawings wherein:

FIG. 1 is a perspective view of an example lead construction comprising a composite material shield layer according to principles of this invention;

FIG. 2 is a transverse cross-sectional view the example embodiment lead construction taken along section A-A illustrating an embodiment comprising the composite material shield layer positioned as an outer layer of the lead construction;

FIG. 3 is a longitudinal cross-sectional view taken along section 3-3 of the example embodiment lead construction of FIG. 2;

FIG. 4 is a transverse cross-sectional view the example embodiment lead construction taken along section A-A illustrating an embodiment comprising the composite material shield layer positioned as an intermediate layer in the lead construction; and

FIG. 5 is a longitudinal view taken along section 5-5 of the example embodiment lead construction of FIG. 4.

### DETAILED DESCRIPTION

Unless otherwise defined, all terms of art, notations and other scientific terms or terminology used herein are intended to have meanings commonly understood by those of skill in the art to which the invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted.

Disclosed herein are composite materials specially formulated from non-ferrous materials to form a conductive shield layer over one or more conductive elements of an implanted medical device such as a cardiac lead when exposed to a high level RF. Such composite material and conductive shield layer is engineered to reduce and/or eliminate RF induced heating of the one or more conductive elements disposed within the lead that may occur during exposure to an MRI procedure. Further, such composite material and conductive shield layer is engineered to provide electrical and/or mechanical properties such as flexibility, electrical insulation, structural integrity, long term fatigue, and the like that are comparable to or that surpasses those found in conventional cardiac leads. In a preferred embodiment, such composite materials are used to form a protective shield layer in an implanted lead used for cardiac applications, neuro applications, or the like.

“Implantable medical device” as used herein may include, but are not limited to ICDs (implantable cardioverter defibillators), pacemakers, leads, catheters, LV lead delivery tools, mechanical valves, stentless tissue valves, stented tissue valves, allografts, repair products, prosthetic devices, subcutaneous and transcutaneous sensors, neurostimulators, cardiac stimulators, or the like. In certain embodiments, the invention pertains to leads, and more specifically, to a protective shield layer that is disposed within the lead. In certain example embodiments of the invention, the shield layer can be an intermediate material layer within the lead and/or can be an outer layer of the lead. The composite materials and shield layers formed therefrom disclosed herein may be adapted for use with variety of implantable medical devices known in the art.

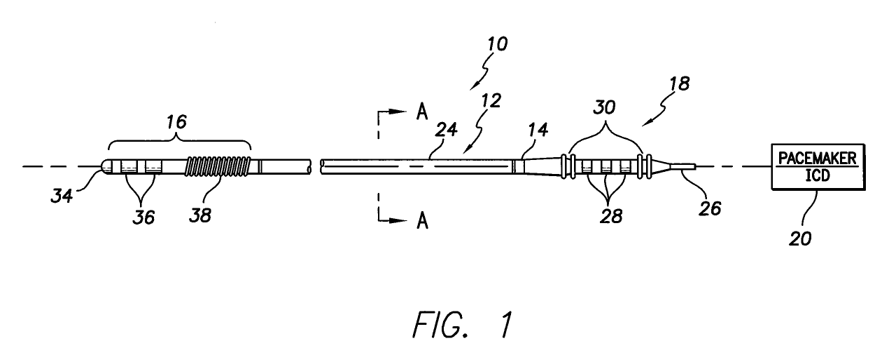

FIG. 1 illustrates an example embodiment cardiac lead 10 constructed according to principles of the invention. The lead 10 has a generally tubular body 12 of given length extending between a proximal end 14 and a distal end 16. A connector assembly 18 extends from the lead proximal end 14 and is adapted to facilitate electrical and mechanical connection with a cardiac-assist device 20, such as a pacemaker, ICD, or the like.

Between the distal end 16 and the proximal end 14, the lead body 12 comprises a flexible insulating sheath or jacket 24, containing one or more conductors, depending on the number of electrodes as disclosed, e.g., in U.S. Pat. Nos. 7,149,578; 6,950,696; 6,934,588; 6,728,579; 6,728,575; 6,615,483; 7,047,073; 6,944,507; 6,931,283; 6,823,215, the contents of each of which are incorporated herein by reference. In one example embodiment, the lead 10 is of a quadripolar design, but in other embodiments the lead will be of a design having a greater or lesser number of poles.

In one embodiment, the lead body 12 may be isodiametric (i.e., the outside diameter of the lead body 12 may be the same throughout its entire length), while in other embodiments the lead body can be configured having a nonconstant diameter. In one embodiment, the outside diameter of the lead body 12 may range from approximately 0.026 inch (2 French) to about 0.130 inch (10 French).

In one embodiment, the connector assembly 18 extends from the proximal end 14 of the lead. The connector assembly 18 can be compatible with a standard such as the IS-4 standard for connecting the lead body to the ICD 20. In an example embodiment, the connector assembly 18 includes a tubular pin terminal contact 26 and ring terminal contacts 28. The connector assembly is received within a receptacle (not shown) in the ICD 20 containing electrical terminals positioned to engage the contacts 26, 28 on the connector assembly 18. As is well known in the art, to prevent ingress of body fluids into the receptacle, the connector assembly 18 is provided with spaced sets of seals 30. In accordance with standard implantation techniques, a stylet or guide wire (not shown) for delivering and steering the distal end of the lead body during implantation is inserted into a lumen of the lead body 12 through the tubular pin terminal contact 26.

In an example embodiment, the distal end 16 of the lead body 12 carries one or more electrodes 34, 36, 38 having configurations, functions and placements along the length of the lead distal end 16 dictated by the desired stimulation therapy, the peculiarities of the patient's anatomy, and so forth. The lead body 12 shown in FIG. 1 illustrates but one example of the various combinations of stimulating and/or sensing electrodes 34, 36, 38 that may be utilized, and it is to be understood that leads having other electrode configurations are within the scope of this invention.

In an example embodiment, the distal end 16 of the lead body 12 includes one tip electrode 34, two ring electrodes 36 and a single cardioverting/defibrillating coil 38. The tip electrode 34 forms the distal termination of the lead body 12. The ring electrodes 36 are positioned adjacent the tip electrode 34. The cardioverter/defibrillator coil 38 is positioned adjacent one of the ring electrodes 36. Depending on the embodiment, the tip and ring electrodes 34, 36 may each serve as tissue-stimulating and/or sensing electrodes.

As noted above, in other embodiments, other electrode arrangements will be employed. For example, in one embodiment, the electrode arrangement may include additional ring stimulation and/or sensing electrodes as well as additional cardioverting and/or defibrillating coils spaced apart along the distal end of the lead body. In one embodiment, the distal end of the lead body may carry only pacing and sensing electrodes, only cardioverting/defibrillating electrodes or a combination of pacing, sensing and cardioverting/defibrillating electrodes.

In conventional fashion, the distal end 16 of the lead body 12 may include passive fixation means (not shown) that may take the form of conventional projecting tines for anchoring the lead body within the right atrium or right ventricle of the heart. Alternatively, the passive fixation or anchoring means may comprise one or more preformed humps, spirals, S-shaped bends, or other configurations manufactured into the distal end 16 of the lead body 12 where the lead is intended for left heart placement within a vessel of the coronary sinus region. The fixation means may also comprise an active fixation mechanism such as a helix or the like. It will be evident to those skilled in the art that any combination of the foregoing fixation or anchoring means may be employed.

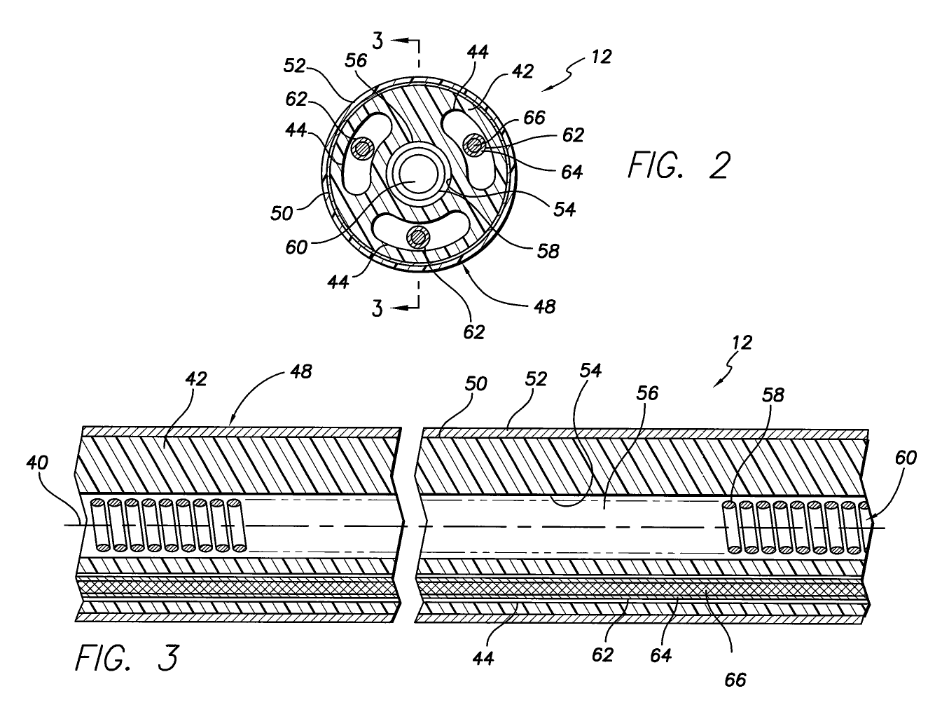

FIG. 2 is a transverse cross-sectional view of the lead tubular body 12 as taken along section line A-A in FIG. 1. FIG. 3 is a longitudinal cross-sectional view of the lead tubular body 12 as taken along section line 3-3 in FIG. 2. As indicated in FIGS. 1 and 3, the lead body 12 extends along a central longitudinal axis 40.

In one embodiment, as illustrated in FIG. 2, the lead construction body includes an insulation layer or wall 42 that in this particular embodiment has three arcuately or radially extending wall lumens 44. In other embodiments, the wall lumen will have other shapes (e.g., square, rectangular, circular, oval, etc.) and/or the insulation wall 42 will have a greater or lesser number of wall lumens 44. In other embodiments, the insulation wall 42 will not have any wall lumens 44.

As indicated in FIGS. 2 and 3, in this particular example embodiment, lead body 12 includes a shield jacket, layer, coating or sheath 48 that extends around an outer surface 50 of the insulation wall 42, and that forms the outer circumferential surface 48 of the lead body 12. The shield layer 48 forming the outer surface of the lead construction is made from a composite material 52 according to principles of this invention. In an example embodiment, the shield layer 48 is formed from a composite material that is engineered to reduce or eliminate the passage of RF signals or energy to conductive elements within the lead, e.g., when the lead is exposed to an MRI procedure.

Example materials useful for forming the shield layer 48 include those materials that are capable of blending with the polymer materials used for making the insulation wall, and that are capable of functioning to shield the conductive elements disposed within the lead covered by the layer from RF signals when exposed thereto. Example materials are additionally ones that are capable of forming an electrical conducting layer when exposed to high RF. Preferred materials useful for forming the shield layer 48 are non-ferrous materials and/or alloys such as gold, platinum, iridium, nickel, cobalt, chromium, molybdenum, alloys, other nonmagnetic materials and/or alloys thereof, carbon/graphite powders, and the like.

In a preferred embodiment, the composite material use to form the shield layer 48 comprises one or more of the above-noted non-ferrous materials that is provided in powder form and that is combined with the polymer material used to form the lead insulation wall 42. Alternatively, the composite material 52 can be combined with a polymer material other than that used to form the lead insulation wall 42, and the resulting composite construction can be disposed over the lead insulation wall outside surface 50. In an example embodiment, the shield layer 48 is a composite material comprising a polymer component such as silicone, medical adhesive and a non-ferrous material such as platinum, iridium, MP35N, silver, and/or gold. The non-ferrous material is preferably provided in the form of particles or powder having a grain size of from about 5 to 70 microns, and more preferably from about 10 to 20 microns.

The density or volume content of the non-ferrous material within the composite material can and will vary depending on the particular end-use application. Generally, the density of the non-ferrous material in the composite material useful shielding RF signals or energy at the high frequencies of say 64 MHz to 128 MHz used for the MRI procedure does not need to be as high as that needed for shielding RF signals or energy used at lower frequencies for other types of operations, such as that used for low frequency pacing/sensing and DF shocks.

In an example embodiment, where the composite material is formed from the preferred polymer and non-ferrous materials noted above, the volume content of the non-ferrous material is in the range of from about 40 to 90 percent, and preferably in the range of from about 50 to 80 percent.

The thickness of the shield layer 48 can and will vary depending on the particular lead construction, the lead application, and the particular materials used to form the shield layer. In an example embodiment, the lead body is constructed comprising a shield layer thickness that provides a resulting lead construction having desired electrical and/or mechanical properties such as electrical insulation, mechanical stiffness, structural rigidity, long term fatigue and the like. The exact thickness of the shield layer will also depend on the configuration of the layer, e.g., whether it is part of the lead insulation wall or whether it is provided as a composite material layer independent of the lead insulation wall. Additionally, the shield layer thickness can vary depending on the materials selected and/or the density of the non-ferrous material used for forming the shield composite material.

In the example embodiment provided above, where the composite material is formed from the preferred polymer and non-ferrous materials noted above, having the volume content of the non-ferrous material noted above, and wherein the shield layer is provided as the outermost region of the lead insulating wall, the shield layer thickness in the wall is in the range of from about 0.1 to 1 mm, and preferably is in the range of from about 0.25 to 0.6 mm. If desired, and in accordance with well-known techniques, the outermost surface of the lead body 12 may also have a lubricious coating or the like along its length to facilitate its movement of the lead through a lead delivery introducer and the patient's vascular system.

Referring again to FIGS. 2 and 3, in an example embodiment, the insulating wall includes an inner circumferential surface 54 that defines a central lumen 56. In one embodiment, a helical coil 58 extends through the central lumen 56 and electrically connects the tubular connector terminal pin 26 with the tip electrode 34. The helical coil 58 defines a coil lumen 60 through which a stylet or guidewire can extend during implantation of the lead.

In one embodiment, the helical coil 58 is a helically coiled multifilar braided cable formed of a metal such as stainless steel, Nitinol, platinum, platinum-iridium alloy, MP35N alloy, MP35N/Ag alloy, or the like. In one embodiment, the helical coil is a helically coiled monofilament or single wire formed of a metal such as stainless steel, Nitinol, platinum, platinum-iridium alloy, MP35N alloy, MP35N/Ag alloy, etc.

In one embodiment, the central lumen 56 does not have a helical coil 58 extending through it. Instead, a liner made of a polymer such as PTFE extends through and lines the central lumen 56. Thus, the central lumen 56 has a slick or lubricious surface for facilitating the passage of the guidewire or stylet through the central lumen 56.

As shown in FIGS. 2 and 3, in one embodiment, each wall lumen 44 includes one or more conductor cables 62 extending through the lumen. In other embodiments wherein the insulation wall 42 does not have any wall lumens 44, the cables 62 will extend through the insulation layer 42 by having the insulation wall 42 co-extruded along the cables 62.

In one embodiment, the conductor cables or wires 62 have a polymer insulation layer or jacket 64. In other embodiments, the conductor cables or wires 62 do not have an insulation layer or jacket. In one embodiment, the core 66 of a conductor cable or wire 62 is a multifilar braided cable formed of a metal such as stainless steel, platinum, platinum-iridium alloy, Nitinol, MP35N alloy, MP35N/Ag alloy, or the like. In one embodiment, the core 66 of a conductor cable or wire 62 is monofilament non-coiled wire formed of a metal such as stainless steel, platinum, platinum-iridium alloy, Nitinol, MP35N alloy, MP35N/Ag alloy, or the like. As can be understood from FIGS. 1, 2 and 3, in one embodiment, two of the cables 62 electrically connect the two of the ring terminal contacts 28 to the two ring electrodes 36, and the third cable 62 electrically connects the third ring terminal contact 28 to the cardioverter/defibrillator coil 38.

The example embodiment lead illustrated in FIGS. 2 and 3, having the conductive material shield layer positioned as an outermost lead surface, can be made in the following manner. The insulating wall 42 of the lead tubular body 12 can be extruded or otherwise formed such that the wall lumens 44 are defined and established in the wall 42 and the wall inner circumferential surface 54 defines the central lumen 56. The desired non-ferrous material used to form the composite material is added to the polymer material forming the insulating wall 42 in a desired amount before the process of extrusion. The helical coil 58 is placed into the central lumen 56, and the conductor cables 62 are placed into their respective wall lumens 44.

In one embodiment, the helical coil 58 is fed into the central lumen 56. In other embodiments, the helical coil 58 is formed into the central lumen 56 or enters the central lumen 56 during extrusion of the wall 42. In one embodiment, the conductor cables 62 are fed into their respective wall lumens 44. In other embodiments, the conductor cables 62 are formed into their respective wall lumens 44 or enter their respective wall lumens 44 during extrusion of the wall 42. In one embodiment, the wall 42 does not have wall lumens for the cables 62 and the cables are formed into their respective locations within the wall 42 during extrusion of the wall 42.

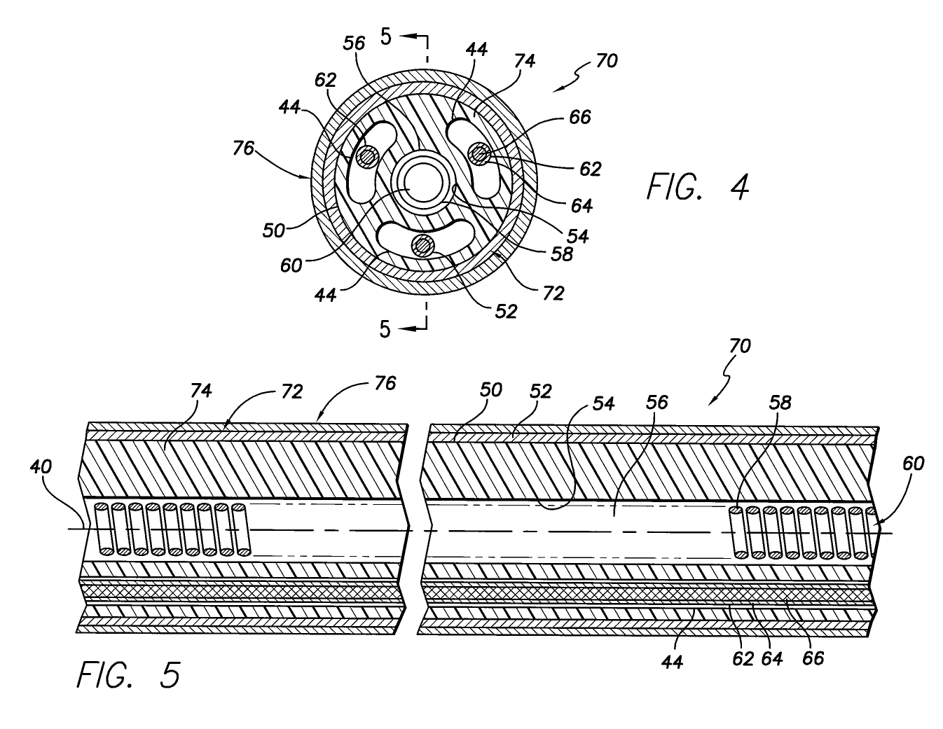

FIGS. 4 and 5 illustrate another example embodiment lead construction 70 of this invention that comprises many of the same elements described above, and such common elements have the same reference numbering as provided in FIGS. 2 and 3. However, this particular embodiment differs in the placement position of the composite material shield layer within the lead construction. Specifically, this example embodiment lead construction 70 comprises a composite material shield layer 72 that is positioned as an intermediate layer within the construction rather than being positioned as an outermost surface of the construction, as illustrated in FIGS. 2 and 3 described above. In this lead construction embodiment, the shield layer 72 occupies an intermediate position interposed between a lead inner insulating wall 74 that is positioned radially inwardly of the shield layer 72, and a lead outer wall structure or layer 76 that is positioned radially outwardly of the shield layer 72.

This example embodiment is provided to demonstrate that lead constructions of this invention can be configured having the shield layer positioned differently within the lead body to provide a desired degree of RF shielding as noted above. Generally, in placing the shield layer within the lead construction, it is desired that an insulating layer be interposed between the shield layer and the one or more conductive elements within the lead. If desired, layers other than and/or in addition to the insulating layer can be interposed between the shield layer and the conductive elements, and further layers can be positioned over the shield layer as called for or as desired to provide a particular lead construction.

While the embodiment illustrated in FIGS. 4 and 5 illustrate placement of the shield layer 72 as an intermediate layer that is positioned around the both the helical coil 58 and the multiple conductors 62, it is to be understood that the shield layer can be positioned differently within the lead construction so as to selectively shield one or more of the conductive elements. For example, the shield layer can be positioned within the lead construction so that it shields the helical coil 58 but not one or more of the conductors 62. Alternatively, the lead construction can be configured having more that one shield layer, e.g., comprising a first shield layer that is positioned within the lead around the helical coil 58, and one or more other shield layers that are positioned within the lead around the one or more respective conductors 62.

The thickness of the shield layer 72 in an example embodiment such as that illustrated in FIGS. 4 and 5, where the shield layer is provided in the form of an intermediate layer, can be the same as described above when it is provided as an outer surface layer of the lead. Additionally, the composite materials used to form such intermediate shield layer can be the same as those described above, and the non-ferrous materials used to form the same can be provided in the same amounts as described above.

While example embodiment lead constructions have been described and illustrated comprising the shield layer as part of the lead body, e.g., as an outer surface layer or as an intermediate layer, it is also to be understood that the shield layer can be embodied differently within the lead construction. For example, the one or more conductive elements disposed within the lead body can be individually coated with an insulating layer and a shield layer, and the lead construction body can then be disposed over such coated conductive elements. The lead construction body in such an alternative embodiment may or may not include further insulating and/or shield layers depending on the particular desired design and performance parameters for the lead construction.

Further, the example embodiment lead constructions have been described and illustrated as having a shield layer that is continuous or that comprises a homogenous distribution of the non-ferrous material with the polymer material. If desired, the shield layer can be discontinuous, e.g., positioned along only partial regions of an underlying conductive element. This can be achieved for example by either selective positioning of multiple shield layers or by forming a nonhomogeneous shield layer where the non-ferrous materials are not uniformly distributed. The extent to which any such shield layer is discontinuous can vary depending on the particular lead construction, but as a general rule would be configured in a manner to provide a desired degree of protection against unwanted RF induced heating of the conductive elements within the lead, e.g., when exposed to an MRI procedure.

In an example embodiment, the shield layer in lead constructions of this invention does not extend to and/or connect with the ring electrode or a header pin of the lead. It is also desired that the shield layer comprise a composite material having the non-ferrous material volume content or density described above to provide an electrically conductive layer capable of shielding high RF frequency signals or energy while also being non-electrically conductive at relatively low RF frequencies signals or energy, e.g., below 64 MHz, to facilitate use of the lead without adversely interfering with such low RF frequency signals that may be used for pacing, sensing, or defibrillation signals.

A feature of lead constructions of this invention is that they comprise one or more shield layer formed from the non-ferrous composite material that is specially formulated to reduce or eliminate unwanted RF induced heating of the electrically conductive elements disposed within the lead, which heating could otherwise travel along the length of the conductive elements and to the electrodes that are attached to the heart wall, e.g., when the lead is a cardiac lead. Such RF induced heating if not otherwise shielded could be sufficient to ablate the interior surface of the blood vessel through which the wire lead is placed, and may be sufficient to cause scarring at the point where the electrodes contact the heart

Additionally, lead constructions of this invention comprising such shield layer formed from the composite materials described herein preferably provide such RF shielding capabilities without sacrificing other desired electrical and/or mechanical performance properties such as electrical insulation, mechanical stiffness, flexibility, structural integrity, and long term fatigue.

Other modifications and variations of lead constructions comprising one or more composite material shield layers of this invention will be apparent to those skilled in the art. It is, therefore, to be understood that within the scope of the appended claims, this invention may be practiced otherwise than as specifically described.

What is claimed is:

1. A lead construction comprising a lead body and one or more conductive elements disposed therein, wherein the lead construction further includes a shield layer that is disposed over at least one of the one or more conductive elements, wherein the shield layer is formed from a composite material comprising a polymer material and a non-ferrous particulate material, wherein the composite material comprises a volume content of the non-ferrous particulate material sufficient to form an electrically conductive layer at RF frequencies greater than about 64 MHz.

2. The lead construction as recited in claim 1 further comprising a layer of insulating material interposed between the shield layer and the at least one or more conductive elements.

3. The lead construction as recited in claim 1 wherein the shield layer is provided as part of the lead body disposed around the one or more conductive elements, and where the shield layer forms an outer surface of the lead body.

4. The lead construction as recited in claim 1 wherein the shield layer is provided as part of the lead body disposed around the one or more conductive elements, and where the shield layer is an intermediate layer within the lead body, and wherein a further layer of insulating material is disposed over the shield layer.

5. The lead construction as recited in claim 1 wherein the non-ferrous material is selected from the group of materials consisting of gold, platinum, iridium, nickel, cobalt, chromium, molybdenum, carbon/graphite powders, and alloys thereof.

6. The lead construction as recited in claim 1 wherein the volume content of the non-ferrous particulate material is such that the composite material does not form an electrically conductive layer at RF frequencies that are lower than about 64 MHz.

7. The lead construction as recited in claim 1 wherein the composite material has a non-ferrous particulate content in the range of from about 40 to 90 volume percent.

8. The lead construction as recited in claim 1 wherein the shield layer has a thickness in the range of from about 0.1 to 1 mm.

9. The lead construction as recited in claim 1 wherein the lead construction is a cardiac lead comprising one or more electrodes positioned adjacent a distal end and connected with the one or more conductive elements disposed within the lead body.

10. A lead construction comprising:
a lead body having a tubular construction defined by distal and proximal body ends, the lead body being formed from a polymeric material;
one or more conductive elements disposed within the lead body;
a shield layer positioned over at least one of the one or more conductive elements, the shield layer being formed from a composite material that is substantially free of a ferrous metal and that forms an electrically conductive layer when exposed to RF having a frequency of greater than about 64 MHz; and
an electrically insulating layer interposed between the at least one or more conductive elements and the shield layer.

11. The lead construction as recited in claim 10 wherein the composite material is formed from one or more polymeric materials and one or more non-ferrous materials.

12. The lead construction as recited in claim 11 wherein the non-ferrous material is selected from the group of materials consisting of gold, platinum, iridium, nickel, cobalt, chromium, molybdenum, carbon/graphite powders, and alloys thereof.

13. The lead construction as recited in claim 10 wherein the composite material has a non-ferrous particulate content in the range of from about 40 to 90 volume percent.

14. The lead construction as recited in claim 10 wherein the shield layer is part of the lead body.

15. The lead construction as recited in claim 14 wherein the shield layer occupies a region of the lead body that is positioned along an outer surface of the lead body.

16. The lead construction as recited in claim 10 wherein the electrically insulating layer is a region of the lead body.

17. The lead construction as recited in claim 10 wherein the shield layer and the electrically insulating layer are both different regions of the lead body.

18. The lead construction as recited in claim 17 wherein the shield layer occupies a region of the lead body positioned intermediate the electrically insulating layer and a further layer of insulating material.

19. The lead construction as recited in claim 10 wherein the shield layer is non-electrically conductive at RF frequencies below about 64 MHz.
